(19) United States
(12) Patent Application Publication
(10) Pub. No.: US 2020/0055731 A1
(43) Pub. Date: Feb. 20, 2020

(54) Novel Process Integration of Methane or Higher Hydrocarbon Pyrolysis Step to Produce Ethylene and Methanol and/or Hydrogen

(71) Applicant: SABIC GLOBAL TECHNOLOGIES, B.V., Bergen op Zoom (NL)

(72) Inventors: Naga Sanyasi Rao VARANASI, Bengaluru (IN); Pankaj Singh GAUTAM, Sugar Land, TX (US); Balamurali NAIR, Sugar Land, TX (US)

(73) Assignee: Sabic Global Technologies, B.V., Bergen op Zoom (NL)

(21) Appl. No.: 16/344,583

(22) PCT Filed: Oct. 25, 2017
(86) PCT No.: PCT/US2017/058208

§ 371 Date: Apr. 24, 2019

Related U.S. Application Data
(60) Provisional application No. 62/413,009, filed on Oct. 26, 2016.

(51) Int. Cl. C01B 3/38; C07C 2/78; C07C 2/84; C07C 29/151; C07C 29/50; C07C 5/09; C07C 5/327

(52) U.S. Cl. CPC C01B 3/386; C07C 2/78; C07C 2/84; C07C 29/151; C07C 11/04; C07C 5/09; C07C 5/327; C01B 2203/0283; C07C 29/50

(57) ABSTRACT

A method for producing ethylene and methanol comprising contacting fuel gas and oxidant gas to produce combustion product; contacting hydrocarbons and combustion product to produce pyrolysis product comprising unconverted hydrocarbons, acetylene, ethylene, CO, H₂, H₂O, CO₂; separating pyrolysis product into CO₂ stream and CO₂ free product comprising unconverted hydrocarbons, acetylene, ethylene, CO, H₂; contacting a first portion of CO₂ free product with aprotic polar solvent to produce acetylene solution and first gas stream comprising unconverted hydrocarbons, ethylene, CO, H₂; contacting acetylene solution with a second portion of CO₂ free product to produce hydrogenation product comprising aprotic polar solvent, unconverted hydrocarbons, ethylene, CO, H₂; separating hydrogenation product into aprotic polar solvent stream and second gas stream comprising unconverted hydrocarbons, ethylene, CO, H₂; separating second gas stream into ethylene stream and third gas stream comprising unconverted hydrocarbons, CO, H₂; and introducing first and/or third gas streams to a reactor to produce methanol.

### CROSS REFERENCE TO RELATED APPLICATIONS

This application is a filing under 35 U.S.C. 371 of International Application No. PCT/US2017/058208 filed Oct. 25, 2017, entitled “Novel Process Integration of Methane or Higher Hydrocarbon Pyrolysis Step to Produce Ethylene and Methanol and/or Hydrogen” which claims priority to U.S. Provisional Application No. 62/413,009 filed Oct. 26, 2016, which applications are incorporated by reference herein in their entirety.

### TECHNICAL FIELD

The present disclosure relates to methods of producing hydrocarbons and alcohols, more specifically methods of producing olefins and methanol by integrating hydrocarbon pyrolysis with methanol production.

### BACKGROUND

Hydrocarbons, and specifically olefins such as ethylene, can be typically used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Methanol can also be used to produce a wide range of products, such as of paints, solvents and plastics, and has found innovative applications in energy, transportation fuel and fuel cells. Methanol is commonly produced from synthesis gas. However, the formation of synthesis gas is strongly endothermic and requires high temperatures, which translates in a high energy input. Thus, there is an ongoing need for the development of processes for the production of olefins such as ethylene, and methanol.

### BRIEF SUMMARY

Disclosed herein is a method for producing ethylene and methanol comprising (a) introducing a first fuel gas stream and an oxidant gas to a combustion zone to produce a combustion product, (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction, (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂), (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂, (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂, (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂, (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂, (h) separating at least a portion of the second gas stream into an ethylene stream and a third gas stream, wherein the third gas stream comprises unconverted hydrocarbons, CO, and H₂, and (i) introducing at least a portion of the first gas stream and/or at least a portion of the third gas stream to a second reaction zone to produce methanol and a second fuel gas stream.

Further disclosed herein is a method for producing ethylene and hydrogen comprising (a) introducing a first fuel gas stream and an oxidant gas to a combustion zone to produce a combustion product, (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction, (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂), (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂, (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂, (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂, (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂, (h) separating at least a portion of the second gas stream into an ethylene stream and a third gas stream, wherein the third gas stream comprises unconverted hydrocarbons, CO, and H₂, and (i) introducing at least a portion of the first gas stream and/or at least a portion of the third gas stream to a pressure swing adsorption (PSA) unit to produce a hydrogen stream and a PSA fuel gas product stream.

Also disclosed herein is a method for producing ethylene, methanol and hydrogen, the method comprising (a) introducing a first fuel gas stream and an oxidant gas to a combustion zone to produce a combustion product, (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction, (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂), (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂, (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂, (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂, (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂, (h) separating at least a portion of the second gas stream into an ethylene stream and a third gas stream, wherein the third gas stream comprises unconverted hydrocarbons, CO, and H₂, (i) introducing a first portion of the combined first gas stream and third gas stream to a second reaction zone to produce methanol and a second fuel gas stream, and (j) introducing a second portion of the combined first gas stream and third gas stream to a pressure swing adsorption (PSA) unit to produce a hydrogen stream and a PSA fuel gas product stream.

### BRIEF DESCRIPTION OF THE DRAWINGS

For a detailed description of the preferred aspects of the disclosed methods, reference will now be made to the accompanying drawing in which:

FIG. 1 displays a schematic of an ethylene and methanol production system;

FIG. 2 displays a schematic of an ethylene and hydrogen production system;

FIG. 3 displays a schematic of an ethylene, methanol, and hydrogen production system; and

FIG. 4 displays a schematic of a pyrolysis experimental system.

### DETAILED DESCRIPTION

Disclosed herein are methods for producing ethylene and methanol comprising (a) introducing a first fuel gas stream and an oxidant gas to a combustion zone to produce a combustion product; (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction; (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂); (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂; (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂; (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂; (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂; (h) separating at least a portion of the second gas stream into an ethylene stream and a third gas stream, wherein the third gas stream comprises unconverted hydrocarbons, CO, and H₂; and (i) introducing at least a portion of the first gas stream and/or at least a portion of the third gas stream to a second reaction zone to produce methanol and a second fuel gas stream. In some aspects, a portion of the combined first gas stream and third gas stream can be introduced to a pressure swing adsorption (PSA) unit to produce a hydrogen stream and a PSA fuel gas product stream.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

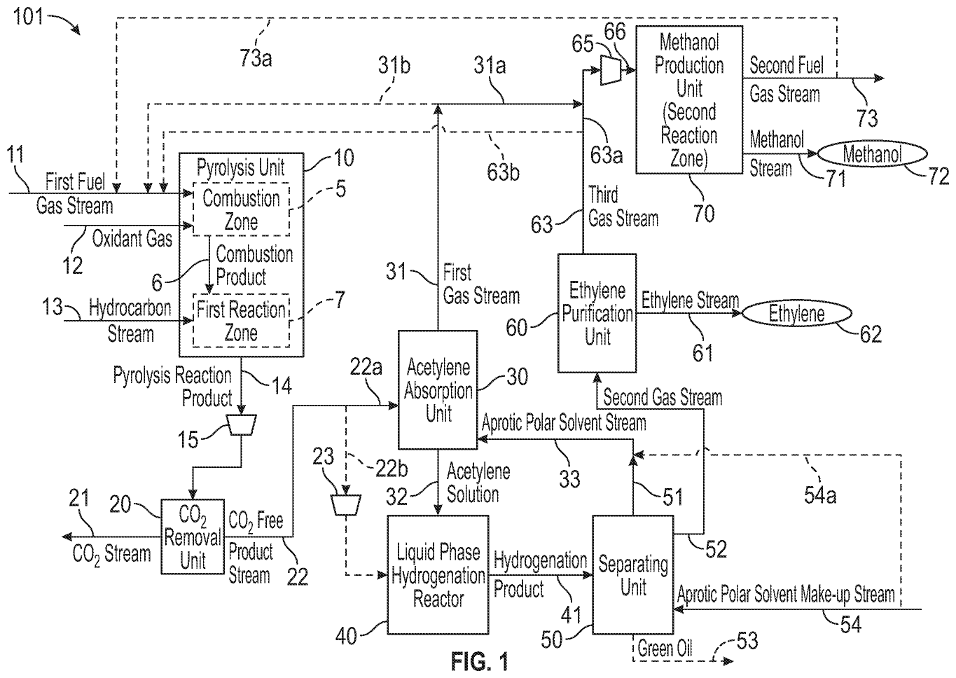

Referring to FIG. 1, an ethylene and methanol production system 101 is disclosed. The ethylene and methanol production system 101 generally comprises a pyrolysis unit 10; a carbon dioxide (CO₂) removal unit 20; an acetylene absorption unit 30; a liquid phase hydrogenation unit 40; a separating unit 50; an ethylene purification unit 60; and a methanol production unit 70.

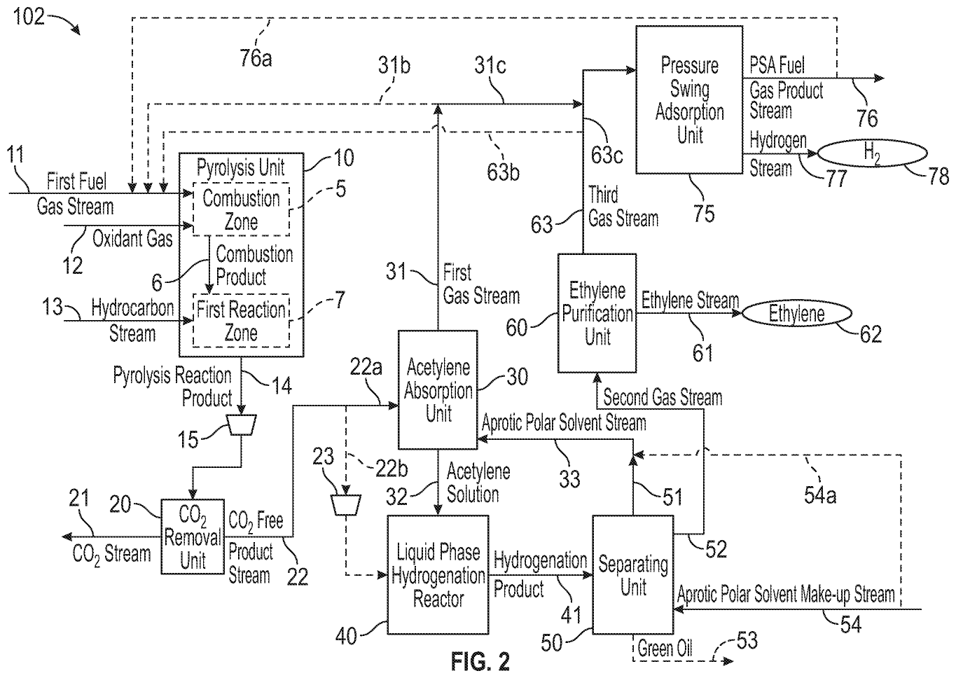

Referring to FIG. 2, an ethylene and hydrogen production system 102 is disclosed. The ethylene and hydrogen production system 102 generally comprises a pyrolysis unit 10; a CO₂ removal unit 20; an acetylene absorption unit 30; a liquid phase hydrogenation unit 40; a separating unit 50; an ethylene purification unit 60; and a pressure swing adsorption (PSA) unit 75.

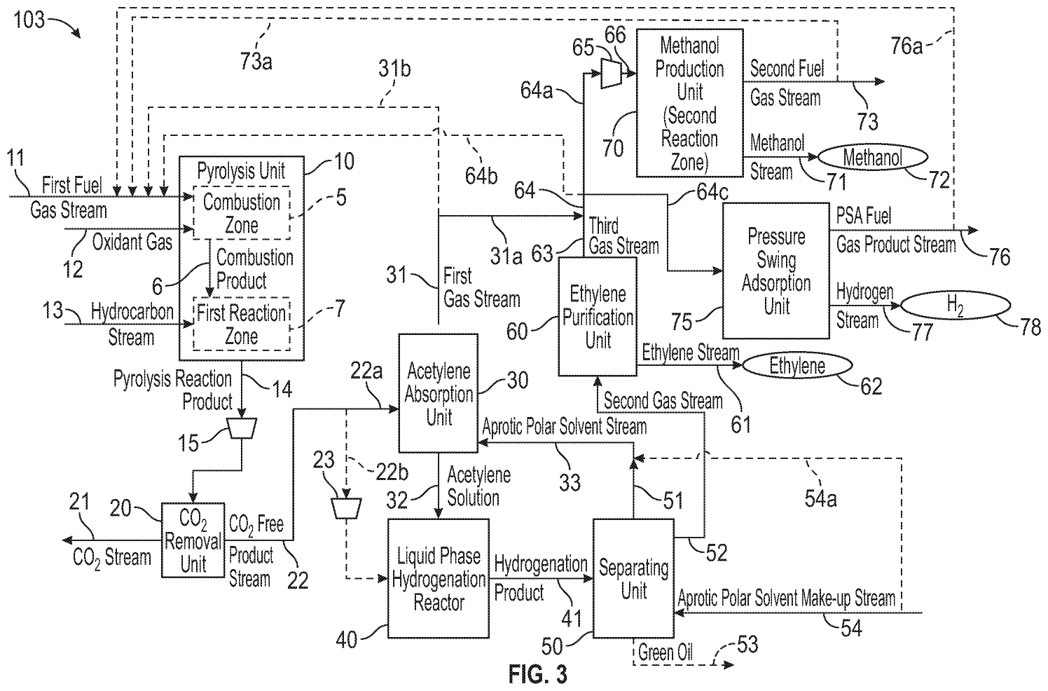

Referring to FIG. 3, an ethylene, methanol, and hydrogen production system 103 is disclosed. The ethylene, methanol, and hydrogen production system 103 generally comprises a pyrolysis unit 10; a CO₂ removal unit 20; an acetylene absorption unit 30; a liquid phase hydrogenation unit 40; a separating unit 50; an ethylene purification unit 60; a methanol production unit 70; and a PSA unit 75. As will be appreciated by one of skill in the art, and with the help of this disclosure, ethylene, and methanol and/or hydrogen production system components shown in FIGS. 1-3 can be in fluid communication with each other (as represented by the connecting lines indicating a direction of fluid flow) through any suitable conduits (e.g., pipes, streams, etc.). Common reference numerals refer to common components present in one or more of the Figures, and the description of a particular component is generally applicable across respective Figures wherein the component is present, expect as otherwise indicated herein.

The pyrolysis unit 10 can comprise a combustion zone 5 and a first reaction zone 7. Impurities and contaminants can be removed from a first fuel gas stream 11 and/or a hydrocarbon stream 13 prior to introducing to the combustion zone 5 and/or the first reaction zone 7, respectively. In some aspects, the first fuel gas stream 11 and the hydrocarbon stream 13 can be the same (e.g., can comprise the same hydrocarbons, for example can be portions of the same gas stream feedstock). In other aspects, the first fuel gas stream 11 and the hydrocarbon stream 13 can be the different (e.g., can comprise different hydrocarbons, for example originating from different upstream sources).

The first fuel gas stream 11 and/or the hydrocarbon stream 13 can comprise methane, natural gas (NG), natural gas liquids, associated gas, well head gas, enriched gas, higher hydrocarbons (e.g., hydrocarbons higher than or having more carbons than methane, C₂₊ hydrocarbons), paraffins, olefins, alcohols, oxygenates, C₁ to C₆ compounds, and the like, or combinations thereof.

In an aspect, a method for producing ethylene, and methanol and/or hydrogen as disclosed herein can comprise a step of introducing the first fuel gas stream 11 and an oxidant gas 12 to the combustion zone 5 to produce a combustion product 6. The combustion zone 5 can comprise a burner, such as an in-line burner; a furnace; or combinations thereof; wherein the first fuel gas stream 11 is burned (e.g., combusted) with the oxidant gas 12 to produce the combustion product 6. The oxidant gas 12 can comprise oxygen, purified oxygen, air, oxygen-enriched air, and the like, or combinations thereof. In some aspects, the oxidant gas 12 is oxygen-enriched, such as oxygen-enriched air, to minimize NOₓ production in the combustion zone 5. As will be appreciated by one of skill in the art, and with the help of this disclosure, NOₓ products can be acidic and as such would necessitate downstream removal. Water or steam can be further introduced to the combustion zone 5 to lower and thereby control the combustion product 6 temperature. The combustion product 6 generally comprises combustion products, such as carbon monoxide (CO), CO₂, water (H₂O), as well as some unconverted hydrocarbons (e.g., hydrocarbons that were present in the first fuel gas stream 11 and did not combust). Depending on the configuration of the pyrolysis unit 10, the combustion product 6 may not be isolatable, and it might be introduced as produced to the first reaction zone 7.

In an aspect, a method for producing ethylene, and methanol and/or hydrogen as disclosed herein can comprise introducing a first reactant mixture to the first reaction zone 7, wherein the first reactant mixture comprises the hydrocarbon stream 13 and at least a portion of the combustion product 6, wherein the hydrocarbon stream 13 comprises natural gas and/or higher hydrocarbons, and wherein the combustion product 6 heats the hydrocarbon stream 13 to a temperature effective for a pyrolysis reaction; and allowing at least a portion of the first reactant mixture to react via the pyrolysis reaction and produce a pyrolysis reaction product 14.

In some aspects, the pyrolysis unit 10 can comprise a reactor that contains both the combustion zone 5 and the first reaction zone 7. In other aspects, the pyrolysis unit 10 can comprise a furnace that contains the combustion zone 5; and a reactor that contains the first reaction zone 7 and is configured to receive the combustion product 6 from the combustion zone 5. A diluent such as an inert gas (e.g., nitrogen, argon, helium, etc.) and/or steam can be further introduced to the first reaction zone 7.

The hydrocarbon stream 13 can be further pre-heated in pre-heaters (e.g., electrical heaters, heat exchangers, etc.) before being heated to a reaction temperature (e.g., temperature effective for a pyrolysis reaction) by direct heat exchange through contact with the combustion product 6. A temperature of the combustion product 6 can be a temperature effective to reach a pyrolysis reaction temperature (e.g., first reaction zone temperature) of equal to or greater than about 700° C., alternatively equal to or greater than about 1,000° C., alternatively equal to or greater than about 1,250° C., alternatively from about 700° C. to about 2,500° C., alternatively from about 1,000° C. to about 2,250° C., or alternatively from about 1,250° C. to about 2,000° C. As will be appreciated by one of skill in the art, and with the help of this disclosure, higher temperatures in the first reaction zone favor alkyne (e.g., acetylene) formation, while lower temperatures in the first reaction zone favor olefin or alkene (e.g., ethylene) formation.

In an aspect, the first reaction zone 7 can be characterized by a residence time effective to allow for the conversion of at least a portion of the first reactant mixture to acetylene and ethylene. The first reaction zone 7 can be characterized by a residence time of from about 0.1 milliseconds (ms) to 100 ms, alternatively from about 0.5 ms to about 80 ms, or alternatively from about 1 ms to about 50 ms.

Suppression or reduction of reactions leading to products other than the desired products (e.g., alkynes, acetylene, olefins, ethylene) may be required to achieve the desired products. This may be accomplished by adjusting the reaction temperature, pressure, and/or quenching after a desired residence time. In some aspects, the hydrocarbon stream 13 that is introduced to the first reaction zone 7 can be characterized by a pressure of from about 1 bar to about 20 bar (e.g., from about 100 kPa to about 2,000 kPa), to achieve the desired products.

The pyrolysis unit 10 can be designed to accommodate one or more gas feed streams (e.g., first fuel gas stream 11, hydrocarbon stream 13), which may employ natural gas combined with other gas components including, but not limited to hydrogen, carbon monoxide, carbon dioxide, ethane, and ethylene. The pyrolysis unit 10 can be designed to accommodate one or more oxidant gas 12 streams, such as an oxygen stream and an oxygen-containing stream for example an air stream, which employ unequal oxidant concentrations for purposes of temperature or composition control. As will be appreciated by one of skill in the art, and with the help of this disclosure, the pyrolysis unit 10 may comprise a single device or multiple devices. Each device of the pyrolysis unit 10 may comprise one or more sections. As illustrated in the figures, products from combustion zone 5 are communicated to the first reaction zone 7 via combustion product stream 6. Depending on the type and configuration of the pyrolysis unit 10 used, stream 6 may not be isolatable (for example, where the combustion zone 5 and the first reaction zone 7 are contained within a common vessel).

In some aspects, to stop pyrolysis reactions occurring in the first reaction zone 7, prevent undesired reverse reactions, or prevent further reactions that form carbon and hydrocarbon compounds other than the desired products, rapid cooling or “quenching” of pyrolysis reaction products can be employed. In an aspect, the pyrolysis unit 10 can further comprise a quench zone, wherein the pyrolysis reaction products are quenched prior to exiting the pyrolysis unit 10 via the pyrolysis reaction product stream 14. The quench zone can employ any suitable quenching methods, for example spraying a quench fluid such as steam, water, oil, or liquid product into a reactor quench zone or chamber; conveying the product stream through or into water, natural gas feed, or liquid products; preheating other streams such as streams 11 and/or 13; generating steam; expanding in a kinetic energy quench, such as a Joule Thompson expander, choke nozzle, turbo expander, etc.; or combinations thereof. As will be appreciated by one of skill in the art, and with the help of this disclosure, the quench zone may be incorporated within a pyrolysis reactor, may comprise a separate vessel or device from the pyrolysis reactor, or both. Pyrolysis units for the production of acetylene and ethylene from hydrocarbons are described in more detail in U.S. Pat. No. 8,445,739 and U.S. Patent Application No. 2010/0167134 A1, each of which is incorporated by reference herein in its entirety.

The pyrolysis reaction product 14 can comprise unconverted hydrocarbons, acetylene, ethylene, CO, H₂, water, and CO₂.

In an aspect, a method for producing ethylene, and methanol and/or hydrogen as disclosed herein can comprise introducing at least a portion of the pyrolysis reaction product 14 to the carbon dioxide removal unit 20 to produce a CO₂ stream 21 and a CO₂ free product stream 22, wherein the CO₂ stream 21 comprises CO₂ and H₂O, and wherein the CO₂ free product stream 22 comprises unconverted hydrocarbons, acetylene, ethylene, CO, and H₂.

In some aspects, the method for producing ethylene, and methanol and/or hydrogen as disclosed herein can further comprise compressing at least a portion of the pyrolysis reaction product 14 (e.g., via compressor 15) to a first pressure range of about 150 psig to about 300 psig, alternatively about 175 psig to about 275 psig, or alternatively about 200 psig to about 250 psig prior to introducing the pyrolysis reaction product 14 to the carbon dioxide removal unit 20. Generally, compressing a gas that contains water from a first pressure to a second pressure (wherein the second pressure is greater than the first pressure) will lead to the water condensing at the second pressure at an increased temperature as compared to a temperature where water of an otherwise similar gas condenses at the first pressure. The carbon dioxide removal unit 20 can comprise a water quench vessel and/or a cooling tower. Compressed gases can be further cooled in the cooling tower (e.g., heat exchanger) and/or in the water quench vessel to promote water condensation and removal.

The carbon dioxide removal unit 20 can comprise a CO₂ separator. In an aspect, at least a portion of CO₂ can be removed from the pyrolysis reaction product 14 by using the CO₂ separator. The CO₂ separator can comprise CO₂ removal by amine (e.g., monoethanolamine) absorption (e.g., amine scrubbing), pressure swing adsorption, temperature swing adsorption, gas separation membranes (e.g., porous inorganic membranes, palladium membranes, polymeric membranes, zeolites, etc.), and the like, or combinations thereof. In an aspect, the CO₂ separator can comprise CO₂ removal by amine absorption.

In an aspect, a method for producing ethylene, and methanol and/or hydrogen as disclosed herein can comprise contacting a first portion 22a of the CO₂ free product stream 22 with an aprotic polar solvent in an acetylene absorption unit 30 to produce an acetylene solution 32 and a first gas stream 31, wherein the aprotic polar solvent absorbs at least a portion of the acetylene of the first portion 22a of the CO₂ free product stream 22 to produce the acetylene solution 32, wherein the acetylene solution 32 comprises at least a portion of the acetylene of the first portion 22a of the CO₂ free product stream 22, and wherein the first gas stream 31 comprises unconverted hydrocarbons, ethylene, CO, and H₂.

The acetylene absorption unit 30 can comprise an acetylene absorption column or tower, wherein at least a portion of the acetylene, and optionally a portion of the ethylene, of the first portion 22a of the CO₂ free product stream 22 is absorbed by the aprotic polar solvent. The aprotic polar solvent can be introduced to the acetylene absorption column via aprotic polar solvent stream 33, which can be introduced co-current with stream 22a; countercurrent with stream 22a; or combinations thereof. The acetylene absorption column can comprise an inert packing material. In some aspects, the acetylene solution 32 can be recovered from the acetylene absorption column as bottoms stream; and the first gas stream 31 can be recovered from the acetylene absorption column as an overhead stream. Nonlimiting examples of aprotic polar solvents suitable for use in the present disclosure include N-methyl-2-pyrrolidone, dimethylformamide, acetone, and the like, or combinations thereof.

In an aspect, the first gas stream 31 can comprise hydrogen in an amount of from about 40 mol % to about 60 mol %, alternatively from about 42.5 mol % to about 57.5 mol %, or alternatively from about 45 mol % to about 55 mol %.

In an aspect, a method for producing ethylene, and methanol and/or hydrogen as disclosed herein can comprise contacting at least a portion of the acetylene solution 32 with a second portion 22b of the CO₂ free product stream 22 in a liquid phase hydrogenation reactor 40 to produce a hydrogenation product 41, wherein the hydrogen of the CO₂ free product stream 22 hydrogenates at least a portion of the acetylene of the acetylene solution 32 to produce ethylene, wherein the hydrogenation product 41 comprises aprotic polar solvent, unconverted hydrocarbons, ethylene, CO, and H₂.

In some aspects, the method for producing ethylene, and methanol and/or hydrogen as disclosed herein can further comprise compressing at least a portion of the second portion 22b of the CO₂ free product stream 22 (e.g., via compressor 23) to a second pressure range of about 200 psig to about 350 psig, alternatively about 225 psig to about 325 psig, or alternatively about 250 psig to about 300 psig prior to introducing the second portion 22b of the CO₂ free product stream 22 to the liquid phase hydrogenation reactor 40.

The liquid phase hydrogenation reactor 40 can be any suitable liquid phase hydrogenation reactor, such as a fixed bed catalytic reactor (typically operated adiabatically); and/or a tubular reactor (typically operated isothermally). Generally, the liquid phase hydrogenation reactor 40 comprises a hydrogenation catalyst, such as a palladium based catalyst, which can be supported on alumina, zeolites, etc. The hydrogenation catalyst can further comprise other metals, such as platinum, silver, nickel, etc.

In an aspect, a method for producing ethylene, and methanol and/or hydrogen as disclosed herein can comprise separating at least a portion of the hydrogenation product 41 into an aprotic polar solvent stream 51 and a second gas stream 52, wherein the aprotic polar solvent stream 51 comprises at least a portion of the aprotic polar solvent of the hydrogenation product 41.

At least a portion of the hydrogenation product 41 can be introduced to the separating unit 50. The separating unit 50 can be any suitable gas liquid separator, such as a vapor liquid separator, flash drum, knock-out drum, knock-out pot, compressor suction drum, etc. The second gas stream 52 can be recovered as an overhead stream, and the aprotic polar solvent stream 51 can be recovered as a bottoms stream. In some aspects, an aprotic polar solvent make-up stream 54 can be introduced to the separating unit 50; combined with streams 51 and/or 33; or combinations thereof as shown by stream 54a to account for any loses of aprotic polar solvent during various process steps, such as hydrogenation, separation, etc. In an aspect, the aprotic polar solvent stream 51 can be recycled to the acetylene absorption unit 30, for example via aprotic polar solvent stream 33. A green oil stream 53 can further be recovered from the separating unit 50, wherein the green oil comprises oligomers that formed in the liquid phase hydrogenation reactor 40.

In an aspect, a method for producing ethylene, and methanol and/or hydrogen as disclosed herein can comprise separating at least a portion of the second gas stream 52 into an ethylene stream 61 comprising ethylene 62 and a third gas stream 63.

The second gas stream 52 comprises unconverted hydrocarbons, ethylene, CO, and H₂. The second gas stream 52 can be characterized by a H₂/CO molar ratio of from about 0.5:1 to about 1.5:1, alternatively from about 0.6:1 to about 1.4:1, or alternatively from about 0.75:1 to about 1.25:1.

At least a portion of the second gas stream 52 can be introduced to the ethylene purification unit 60 to produce the ethylene stream 61 and the third gas stream 63. The ethylene purification unit 60 can employ a variety of separation processes, such as cryogenic distillation.

The third gas stream 63 comprises unconverted hydrocarbons, CO, and H₂. The third gas stream 63 can be characterized by a H₂/CO molar ratio of from about 0.5:1 to about 1.5:1, alternatively from about 0.6:1 to about 1.4:1, or alternatively from about 0.75:1 to about 1.25:1. As will be appreciated by one of skill in the art, and with the help of this disclosure, the H₂/CO molar ratio of the second gas stream 52 and the third gas stream 63 are about the same, as the ethylene purification process does not alter substantially the H₂/CO molar ratio.

As will be appreciated by one of skill in the art, and with the help of this disclosure, by removing ethylene from the second gas stream 52, all other components remaining in the gas stream, such as hydrogen, are more concentrated. In an aspect, the third gas stream can comprise hydrogen in an amount of from about 25 mol % to about 40 mol %, alternatively from about 27.5 mol % to about 37.5 mol %, or alternatively from about 30 mol % to about 35 mol %.

In an aspect, a method for producing ethylene, and methanol and/or hydrogen as disclosed herein can comprise introducing at least a portion of the first gas stream 31 and/or at least a portion of the third gas stream 63 to a second reaction zone (e.g., methanol production unit 70) to produce a methanol stream 71 comprising methanol 72 and a second fuel gas stream 73, wherein the second fuel gas stream 73 comprises hydrocarbons (e.g., unconverted hydrocarbons), ethylene, or combinations thereof.

The first gas stream 31 can be characterized by a H₂/CO molar ratio of from about 1.5:1 to about 3.0:1, alternatively from about 1.6:1 to about 2.9:1, or alternatively from about 1.75:1 to about 2.75:1. As will be appreciated by one of skill in the art, and with the help of this disclosure, the methanol production unit 70 has specific H₂/CO molar ratio requirements, and as such the first gas stream 31 and the third gas stream 63 can be combined in a proportion effective to provide for the specific H₂/CO molar ratio requirement of the methanol production unit 70 (e.g., H₂/CO molar ratio of about 2:1).

In an aspect, the methanol production unit 70 can be characterized by an M ratio requirement of from about 2.0 to about 2.2, or alternatively from about 2.05 to about 2.15. For purposes of the disclosure herein, the M ratio is a molar ratio defined as (H₂—CO₂)/(CO+CO₂).

In aspects where a feed stream to the methanol production unit 70 (e.g., at least a portion of the first gas stream 31 and/or at least a portion of the third gas stream 63) is characterized by an M ratio other than from about 2.0 to about 2.2, at least a portion of the feed stream to unit 70 can be subjected to a water-gas shift reaction to produce a shifted gas stream, wherein the shifted gas stream is characterized by an M ratio of from about 2.0 to about 2.2. In such aspect, the shifted gas stream can be introduced to the methanol production unit 70 to produce methanol. Generally, the water-gas shift reaction describes the catalytic reaction of carbon monoxide and water vapor to form carbon dioxide and hydrogen according to the reaction CO+H₂O [symbol] CO₂+H₂. Typically, the water-gas shift reaction is used to increase the H₂/CO molar ratio of gas streams comprising carbon monoxide and hydrogen. As will be appreciated by one of skill in the art, and with the help of this disclosure, gas streams comprising hydrogen and CO can be referred to as synthesis gas. Water-gas shift catalysts can comprise any suitable water-gas shift catalysts, such as commercial water-gas shift catalysts; chromium or copper promoted iron-based catalysts; copper-zinc-aluminum catalyst; and the like; or combinations thereof.

In aspects where a feed stream to the methanol production unit 70 (e.g., at least a portion of the first gas stream 31 and/or at least a portion of the third gas stream 63) is characterized by methane content of equal to or greater than about 3 mol %, alternatively equal to or greater than about 4 mol %, or alternatively equal to or greater than about 5 mol %, at least a portion of the feed stream to unit 70 can be subjected to a methane steam reforming reaction to produce a synthesis gas stream, wherein the synthesis gas stream is characterized by methane content of less than about 3 mol %, alternatively less than about 2 mol %, or alternatively less than about 1 mol %. In such aspect, the synthesis gas stream can be introduced to the methanol production unit 70 to produce methanol. Generally, the steam methane reforming describes the catalytic reaction of methane and steam to form carbon monoxide and hydrogen according to the reaction CH₄+H₂O [symbol] CO+3H₂. Typically, the steam methane reforming reaction is used to decrease the methane content of the gas streams entering the methanol production unit 70. The steam reforming catalysts can comprise any suitable commercially available steam reforming catalyst; nickel (Ni) and/or rhodium (Rh) as active metal(s) on alumina; or combinations thereof.

The methanol production unit 70 can comprise any reactor suitable for a methanol synthesis reaction from CO and H₂, such as for example an isothermal reactor, an adiabatic reactor, a slurry reactor, a cooled multi tubular reactor, and the like, or combinations thereof.

In an aspect, at least a portion of the CO and at least a portion of the H₂ of a feed stream to the methanol production unit 70 (e.g., at least a portion of the first gas stream 31 and/or at least a portion of the third gas stream 63) can undergo a methanol synthesis reaction. Generally, CO and H₂ can be converted into methanol (CH₃OH) according to reaction CO+2H₂═CH₃OH. Methanol synthesis from CO and H₂ is a catalytic process, and is most often conducted in the presence of copper based catalysts.

The methanol production unit 70 can comprise a catalyst, such as any suitable commercial catalyst used for methanol synthesis. Nonlimiting examples of catalysts suitable for use in the methanol production unit 70 in the current disclosure include Cu, Cu/ZnO, Cu/ThO₂, Cu/Zn/Al₂O₃, Cu/ZnO/Al₂O₃, Cu/Zr, and the like, or combinations thereof.

The methanol production unit 70 can be characterized by a second reaction zone temperature of from about 150° C. to about 400° C., alternatively from about 165° C. to about 300° C., or alternatively from about 180° C. to about 250° C. The methanol production unit 70 can be characterized by a pressure of from about 1,000 psig to about 1,300 psig, alternatively from about 1,050 to about 1,250 psig, or alternatively from about 1,100 to about 1,200 psig.

In an aspect, a method for producing ethylene and methanol as disclosed herein can comprise recovering a CH₃OH stream 71 from the methanol production unit 70, for example by flashing. In an aspect, CH₃OH stream 71 comprises CH₃OH, H₂O and heavy alcohols (e.g. C₂₊ alcohols). In an aspect, a method for producing ethylene and methanol can further comprise recovering CH₃OH 72 from the CH₃OH stream 71, for example by distillation.

In some aspects, the feed stream to the methanol production unit 70 (e.g., at least a portion of the first gas stream 31 and/or at least a portion of the third gas stream 63) can be pressurized to a pressure of from about 1,000 psig to about 1,300 psig prior to introducing to the methanol production unit 70.

As illustrated in the configuration of the ethylene and methanol production system 101 in FIG. 1, at least a portion 31a of the first gas stream 31 and/or at least a portion 63a of the third gas stream 63 can be compressed (e.g., via compressor 65) to a third pressure range of about 1,000 psig to about 1,300 psig, alternatively about 1,050 psig to about 1,250 psig, or alternatively about 1,100 psig to about 1,200 psig to produce a compressed gas stream 66. In an aspect, at least a portion of the compressed gas stream 66 can be introduced to the second reaction zone (e.g., methanol production unit 70), wherein the compressed gas stream is characterized by an M ratio of from about 2.0 to about 2.2.

As illustrated in the configuration of the ethylene, methanol, and hydrogen production system 103 in FIG. 3, at least a portion 31a of the first gas stream 31 can be combined with at least a portion of the third gas stream 63 to produce a fourth gas stream 64. In an aspect, a first portion 64a of the fourth gas stream 64 can be compressed (e.g., via compressor 65) to a third pressure range of about 1,000 psig to about 1,300 psig, alternatively about 1,050 psig to about 1,250 psig, or alternatively about 1,100 psig to about 1,200 psig to produce the compressed gas stream 66. In such aspect, at least a portion of the compressed gas stream 66 can be introduced to the second reaction zone (e.g., methanol production unit 70), wherein the compressed gas stream is characterized by an M ratio of from about 2.0 to about 2.2.

In aspects where the compressed gas stream 66 is characterized by an M ratio other than from about 2.0 to about 2.2, at least a portion of the compressed gas stream is subjected to a water-gas shift reaction to produce a shifted gas stream characterized by an M ratio of from about 2.0 to about 2.2. In such aspects, at least a portion of the shifted gas stream can be introduced to the methanol production unit 70 to produce methanol.

In an aspect, a method for producing ethylene, and methanol and/or hydrogen as disclosed herein can comprise introducing at least a portion of the first gas stream 31 and/or at least a portion of the third gas stream 63 to a pressure swing adsorption (PSA) unit 75 to produce a hydrogen stream 77 comprising hydrogen 78 and a PSA fuel gas product stream 76, wherein the PSA fuel gas product stream 76 comprises hydrocarbons (e.g., unconverted hydrocarbons), ethylene, or combinations thereof. Generally, hydrogen can be recovered from gas streams by using a PSA process which is based on a physical binding of gas molecules to adsorbent material, wherein forces acting between gas molecules and adsorbent material depend on the gas component, type of adsorbent material, partial pressure of the gas component and operating temperature. The separation effect is based on differences in binding forces to the adsorbent material. Highly volatile components with low polarity, such as hydrogen, are practically non-adsorbable, as opposed to molecules as N₂, CO, CO₂, hydrocarbons and water vapor, and as such high purity hydrogen can be recovered.

As illustrated in the configuration of the ethylene and hydrogen production system 102 in FIG. 2, at least a portion 31c of the first gas stream 31 and/or at least a portion 63c of the third gas stream 63 can be introduced to the PSA unit 75 to produce hydrogen.

As illustrated in the configuration of the ethylene, methanol, and hydrogen production system 103 in FIG. 3, a second portion 64c of the fourth gas stream 64 can be introduced to the PSA unit 75 to produce hydrogen.

As illustrated in the configuration of the ethylene and methanol production system 101 in FIG. 1, a portion 31b of the first gas stream 31, a portion 63b of the third gas stream 63, a portion 73a of the second fuel gas stream 73, or combinations thereof can be recycled to the combustion zone 5, for example via the first fuel gas stream 11. In some aspects, the portion 31b of the first gas stream 31, the portion 63b of the third gas stream 63, the portion 73a of the second fuel gas stream 73, or combinations thereof can be used as a fuel stream other than the first fuel gas stream 11.

As illustrated in the configuration of the ethylene and hydrogen production system 102 in FIG. 2, a portion 31b of the first gas stream 31, a portion 63b of the third gas stream 63, a portion 76a of the PSA fuel gas product stream 76, or combinations thereof can be recycled to the combustion zone 5, for example via the first fuel gas stream 11. In some aspects, the portion 31b of the first gas stream 31, the portion 63b of the third gas stream 63, the portion 76a of the PSA fuel gas product stream 76, or combinations thereof can be used as a fuel stream other than the first fuel gas stream 11.

As illustrated in the configuration of the ethylene, methanol, and hydrogen production system 103 in FIG. 3, a portion 31b of the first gas stream 31, a portion 64b of the fourth gas stream 64 (comprising a portion of the first gas stream 31 and a portion of the third gas stream 63), a portion 73a of the second fuel gas stream 73, a portion 76a of the PSA fuel gas product stream 76, or combinations thereof can be recycled to the combustion zone 5, for example via the first fuel gas stream 11. In some aspects, the portion 31b of the first gas stream 31, the portion 64b of the fourth gas stream 64, the portion 73a of the second fuel gas stream 73, the portion 76a of the PSA fuel gas product stream 76, or combinations thereof can be used as a fuel stream other than the first fuel gas stream 11.

In an aspect, a method for producing ethylene, and methanol and/or hydrogen as disclosed herein can comprise (a) introducing a first portion of a hydrocarbon stream and an oxidant gas to a combustion zone to produce a combustion product, wherein the hydrocarbon stream comprises natural gas and/or higher hydrocarbons; (b) introducing a first reactant mixture to a first reaction zone, wherein the first reactant mixture comprises a second portion of the hydrocarbon stream and at least a portion of the combustion product, and wherein the combustion product heats the second portion of the hydrocarbon stream to a temperature of equal to or greater than about 700° C.; (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂); (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂; (e) contacting a first portion of the CO₂ free product stream with dimethylformamide (DMF) in an acetylene absorption unit to produce an acetylene solution and a first gas stream, wherein the DMF absorbs at least a portion of the acetylene of the first portion of the CO₂ free product stream to produce the acetylene solution, wherein the acetylene solution comprises at least a portion of the acetylene of the first portion of the CO₂ free product stream, wherein the first gas stream comprises unconverted hydrocarbons, ethylene, CO, and H₂, and wherein the first gas stream has a H₂/CO molar ratio of from about 1.5:1 to about 3.0:1; (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises DMF, unconverted hydrocarbons, ethylene, CO, and H₂; (g) separating at least a portion of the hydrogenation product into a DMF stream and a second gas stream, wherein the DMF stream comprises at least a portion of the DMF of the hydrogenation product, wherein the second gas stream comprises unconverted hydrocarbons, ethylene, CO, and H₂, and wherein the second gas stream can be characterized by a H₂/CO molar ratio of from about 0.5:1 to about 1.5:1; (h) separating at least a portion of the second gas stream into an ethylene stream comprising ethylene and a third gas stream, wherein the third gas stream comprises unconverted hydrocarbons, CO, and H₂, and wherein the third gas stream comprises hydrogen in an amount of from about 25 mol % to about 40 mol %; and (i) introducing a first portion of the combined first gas stream and third gas stream to a second reaction zone to produce methanol and a second fuel gas stream, wherein the second fuel gas stream hydrocarbons (e.g., unconverted hydrocarbons), ethylene, or combinations there, and/or (j) introducing a second portion of the combined first gas stream and third gas stream to a pressure swing adsorption (PSA) unit to produce a hydrogen stream and a PSA fuel gas product stream, and wherein the PSA fuel gas product stream comprises hydrocarbons (e.g., unconverted hydrocarbons), ethylene, or combinations there. In some aspects, the method for producing ethylene, and methanol and/or hydrogen as disclosed herein can further comprise recycling a portion of the first gas stream, a portion of the third gas stream, at least a portion of the second fuel gas stream, at least a portion of the PSA fuel gas product stream, or combinations thereof to the combustion zone as the first fuel gas stream. In aspects where the first portion of the combined first gas stream and third gas stream is characterized by an M ratio other than from about 2.0 to about 2.2, at least a portion of the first portion of the combined first gas stream and third gas stream can be subjected to a water-gas shift reaction to produce a shifted gas stream, wherein the shifted gas stream is characterized by an M ratio of from about 2.0 to about 2.2. In such aspects, the shifted gas stream can be introduced to the second reaction zone to produce methanol.

In an aspect, a method for producing ethylene, and methanol and/or hydrogen as disclosed herein can advantageously display improvements in one or more method characteristics when compared to an otherwise similar method that does not integrate hydrocarbon pyrolysis with other processes for producing desired products. A synthesis gas (e.g., H₂ and CO) to methanol conversion process as disclosed herein can increase further the overall efficiency of the process by producing methanol from the H₂ and CO obtained from hydrocarbon pyrolysis. PSA recovery of hydrogen can increase further the overall efficiency of the process. For example, the hydrogen recovered via PSA can be further used in a variety of processes, such as ammonia production, hydrodesulfurization, etc.

In an aspect, a method for producing ethylene, and methanol and/or hydrogen as disclosed herein can advantageously display an increased overall carbon efficiency when compared to a carbon efficiency of a similar hydrocarbon pyrolysis process that is not integrated with synthesis gas to methanol conversion. In such aspect, the increased overall carbon efficiency of the method can be due to using a new integration scheme of hydrocarbon pyrolysis with acetylene hydrogenation and methanol production by taking advantage of conversion of large amounts of CO and hydrogen formed in the hydrocarbon pyrolysis to additional valuable products such as methanol. The methanol can be advantageously used as a liquid fuel, and can be easily transported, as compared to transporting gases. Additional advantages of the methods for producing ethylene, and methanol and/or hydrogen as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

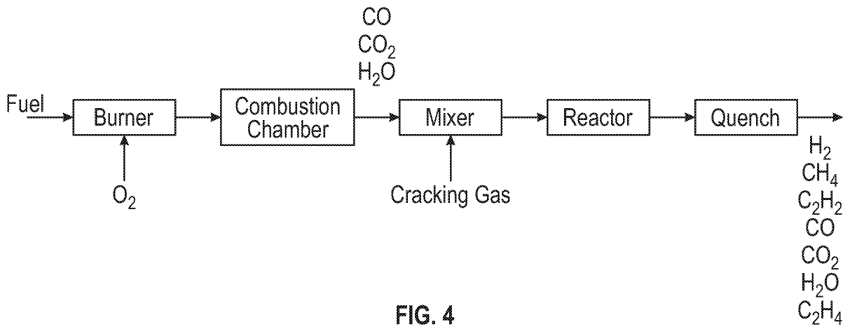

A cracking or pyrolysis experimental system as illustrated in FIG. 4 was used to further investigate the methanol and/or hydrogen production systems disclosed herein.

The pyrolysis experimental system (FIG. 4) encompassed four steps: (i) combusting of fuel gases in a combustion chamber; (ii) mixing of cracking feed (natural gas (NG)/field gas) with products of the combustion in a mixing or mixer section; followed by (iii) cracking or pyrolysis of the above mixture (produced in step (iii)) in a reactor section; and (iv) quenching the products from the reactor section. The combustion chamber produced hot gases with a temperature of about 2,500° C. These hot gases were mixed with feed natural gas (e.g., cracking gas), which was optionally preheated (300-500° C.). The combustion gases transferred heat to the feed natural gas by direct contact, and the feed further underwent pyrolysis in the reactor section. Major products of the pyrolysis included acetylene (C₂H₂), ethylene (C₂H₄), and hydrogen (H₂). However carbon monoxide (CO), carbon dioxide (CO₂), and water (H₂O) were also formed, mostly from the combustion chamber.

The reactor section comprised a high-temperature, high-velocity, water-cooled thermal reactor that dehydrogenated (cracked) the hydrocarbon feed. The typical feed to the reactor (e.g., cracker) was natural gas, which was converted to alkene and alkyne products. The generalized, simplified global reaction sequence that took place in the reactor section can be represented as depicted in reactions (1)-(5):

2CH₄→C₂H₆+H₂ (1)

C₂H₆→C₂H₄+H₂ (2)

C₂H₄→C₂H₂+H₂ (3)

3C₂H₂→C₆H₆ (4)

C₆H₆→PAH(Coke)+H₂ (5)

wherein PAH=polyaromatic hydrocarbon.

All of the above reactions ((1)-(5)) are endothermic, and hence their products are favored thermodynamically at high temperature. Given sufficient residence time, all products would be coke and hydrogen. Neglecting the coke formation during methane pyrolysis, the global reaction of pyrolysis can be written as:

2CH4→C₂H₂+3H₂ ΔH°=189 kJ/mol (6)

The endothermic pyrolysis or cracking reaction requires 189 kJ per mol of CH₄. This energy was supplied by an oxy-fuel combustion process. The simplified reaction sequence for the combustion (typically methane) can be represented as depicted in reactions (7)-(9):

CH₄+½O₂→CO+2H₂ ΔH°=−36 kJ/mol (7)

H₂+½O₂→H₂O ΔH°=−242 kJ/mol (8)

CO+½O₂→CO₂ ΔH°=−283 kJ/mol (9)

These reactions ((7)-(9)) are homogeneous radical reactions. The reactions (7)-(9) do not go to completion, and conversion is limited by residence time and temperature.

The operation of the quench section was an integral part of the pyrolysis reactor. The quench design allowed for control of pyrolysis reaction zone and residence time. To control the residence time of reaction (to stop the reaction) in order to obtain the desired product (acetylene), liquid coolant, typically water, was directly sprayed into the gas stream produced by the reactor section. The amount of quench used depended on pyrolysis reactor heat output. The quench nozzle was located in the center of the effluent stream. The coolant and effluent flow were introduced countercurrent into the quench section. Coolant was also utilized in the decoking of the pyrolysis reactor. The expected temperature at the exit of the quench section was 200 to 300° F.

Gas samples were collected after the quench section and were fed into a gas chromatograph (GC). The GC output was processed to yield mole fractions values of chemical species including the desired product, acetylene. The compositions in mole fractions were further transformed into more tangible performance indices of the pyrolysis unit, such as yield of C₂H₂, conversion of cracker feed or natural gas feed to reactor, and selectivity of C₂H₂.

The H₂ rich gas from the reactor was further washed in a spray tower to remove coke/carbon fines, and was followed by an amine column to remove CO₂. The gases then went through a compression step and were split into two streams, with approximately an 1:3 ratio by weight, with the larger amount of gas going to an absorption step, and the smaller amount of gas going to a hydrogenation step, wherein the smaller amount of gas fulfilled the role of a H₂ source. The absorption of acetylene was carried out with a solvent, N-methyl pyrrolidone (NMP) at 125 psig and 40° C. NMP is a fairly common industrial solvent used to recover pure hydrocarbons, owing to its relatively low volatility, low flammability, and relatively low toxicity. The solubility of C₂H₂ in NMP is limited to <5 wt. % in NMP under normal operating conditions. NMP with dissolved acetylene was sent to a hydrogenation reactor. Vent gases from the absorber were sent to the combustion chamber of the pyrolysis section as a fuel gas.

The conversion of acetylene to ethylene was accomplished in a trickle bed reactor, over a proprietary Pd—Zn/α-Al₂O₃ catalyst (0.5 wt. % each metal; alumina supported palladium and zinc based catalyst). The goal of the hydrogenation piloting runs was to study the conversion of acetylene to ethylene, and the selectivity to ethylene as a function of the liquid hourly space velocity (LHSV), the weight hourly space velocity (WHSV), and the reactor inlet temperature, for the different feedstocks. The hydrogenation reactor was operated at 250 psig and around 90° C. The products were subsequently depressurized in another unit to produce crude ethylene, which could then be purified to make polymer grade ethylene. The solvent NMP was recycled to the absorption column after a simple purification step. A combination of the use of liquid phase, limited solubility of C₂H₂ in solvent, and partial deactivation of catalyst by CO enabled safe and controlled operation of the hydrogenation reactor. The limited solubility of C₂H₂ in NMP helped reduce the availability of C₂H₂ for the liquid phase reaction. The reactor operated at 1.4 wt. % of C₂H₂ dissolved in NMP, which along with the use of liquid phase hydrogenation helped dissipate the heat of the reaction. A certain amount of CO in the H₂ rich gas stream helped reduce the number of active sites in the Pd catalyst, which also played a role in controlling the reaction from runaway situations.

The composition of various gas streams of the ethylene, and methanol and/or hydrogen production systems as illustrated in FIGS. 1-4 was investigated, and is displayed in Tables 1 and 2.

TABLE 1
First Gas Stream composition for natural gas (NG) feed

| Component | Mol % |
|---|---|
| H2 | 48.6 |
| CO2 | 1.0 |
| CH4 | 13.3 |
| C2H4 | 0.9 |
| C3H6 | 0.03 |
| C2H2 | 0.53 |
| C3H4 | 0.05 |
| CO | 34.3 |
| C4H6 | 0.09 |
| C6s | 0.01 |
| N2 | 1.0 |
| O2 | 0.2 |

TABLE 2
Second Gas Stream composition for natural gas (NG) feed

| Component | Mol % |
|---|---|
| H2 | 32.0 |
| CO2 | 1.8 |
| CH4 | 13.7 |
| C2H6 | 0.18 |
| C2H4 | 14.9 |
| C3H6 | 0.01 |
| C2H2 | 1.3 |
| C3H4 | 0.17 |
| C4 olefins | 0.05 |
| CO | 34.1 |
| C4H6 | 0.4 |
| C6s | 0.1 |
| N2 | 1.1 |
| O2 | 0.16 |

The data in Table 1 provide a typical composition of the first gas stream 31 produced in the ethylene, and methanol and/or hydrogen production systems as disclosed herein. The first gas stream 31 can be mixed with fuel gas from the hydrocarbon separation to provide the desired H₂/CO molar ratio (about 2.0:1) for the methanol synthesis or H₂ feed streams (third gas stream 63) for hydrogen separation and recovery. The data in Table 2 provide a typical composition of the second gas stream 52 produced in the ethylene, and methanol and/or hydrogen production systems as disclosed herein.

Additional compositions of the first gas stream 31 and the second gas stream 52 (from experimental data) produced in the ethylene, and methanol and/or hydrogen production systems as disclosed herein for different hydrocarbon feeds (e.g., hydrocarbon stream 13), are given in Tables 3, 4 and 5. The feeds (e.g., hydrocarbon stream 13) included a mixture of natural gas (NG) and propane; a mixture of NG and pentane; and a mixture of NG and hexane; for the data displayed in Tables 3, 4 and 5, respectively.

TABLE 3
NG (66 mol %) & propane (34 mol %) feed mix

| | First gas stream | Second gas stream |
|---|---|---|
| Hydrogen | 46.161 | 18.940 |
| Carbon Dioxide | 5.363 | 12.824 |
| Oxygen/Argon | 0.000 | 0.099 |
| Methane | 14.548 | 14.186 |
| Nitrogen | 0.435 | 0.426 |
| Ethane | 0.024 | 0.235 |
| Ethylene | 1.925 | 22.268 |
| Propane | 0.000 | 0.000 |
| Propylene | 0.024 | 0.167 |
| Acetylene | 0.162 | 2.025 |
| Isobutane | 0.000 | 0.000 |
| N-Butane | 0.000 | 0.000 |
| Propadiene | 0.000 | 0.084 |
| C4 Olefins | 0.021 | 0.204 |
| Carbon Monoxide | 31.298 | 27.965 |
| Isopentane | 0.000 | 0.000 |
| N-Pentane | 0.000 | 0.000 |
| Methyl Acetylene | 0.000 | 0.002 |
| 1,3-Butadiene | 0.040 | 0.502 |
| C5s | 0.000 | 0.000 |
| C6s | 0.000 | 0.073 |

TABLE 4
NG (86 mol %) & pentane (14 mol %) feed mix

| | First gas stream | Second gas stream |
|---|---|---|
| Hydrogen | 47.05 | 26.18 |
| Carbon Dioxide | 0.00 | 0.06 |
| Oxygen/Argon | 0.31 | 0.25 |
| Methane | 16.05 | 16.37 |
| Nitrogen | 0.97 | 0.98 |
| Ethane | 0.04 | 0.21 |
| Ethylene | 1.79 | 18.94 |
| Propane | 0.00 | 0.00 |
| Propylene | 0.02 | 0.09 |
| Acetylene | 0.45 | 3.65 |
| Isobutane | 0.00 | 0.00 |
| N-Butane | 0.00 | 0.00 |
| Propadiene | 0.05 | 0.32 |
| C4 Olefins | 0.00 | 0.05 |
| Carbon Monoxide | 33.25 | 32.75 |
| Isopentane | 0.00 | 0.00 |
| N-Pentane | 0.00 | 0.00 |
| Methyl Acetylene | 0.00 | 0.00 |
| 1,3-Butadiene | 0.00 | 0.00 |
| C5s | 0.00 | 0.00 |
| C6s | 0.01 | 0.14 |

TABLE 5
NG (78 mol %) & hexane (22 mol %) feed mix

| | First gas stream | Second gas stream |
|---|---|---|
| Hydrogen | 45.43 | 22.13 |
| Carbon Dioxide | 0.00 | 0.08 |
| Oxygen/Argon | 0.21 | 0.14 |
| Methane | 17.69 | 17.84 |
| Nitrogen | 1.02 | 0.97 |
| Ethane | 0.06 | 0.64 |
| Ethylene | 3.05 | 21.40 |
| Propane | 0.00 | 0.00 |
| Propylene | 0.04 | 0.19 |
| Acetylene | 0.42 | 4.56 |
| Isobutane | 0.00 | 0.00 |
| N-Butane | 0.00 | 0.00 |
| Propadiene | 0.06 | 0.39 |
| C4 Olefins | 0.01 | 0.10 |
| Carbon Monoxide | 31.99 | 31.37 |
| Isopentane | 0.00 | 0.00 |
| N-Pentane | 0.00 | 0.00 |
| Methyl Acetylene | 0.00 | 0.00 |
| 1,3-Butadiene | 0.00 | 0.00 |
| C5s | 0.00 | 0.00 |
| C6s | 0.01 | 0.20 |

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first aspect, which is a method for producing ethylene and methanol comprising (a) introducing a first fuel gas stream and an oxidant gas to a combustion zone to produce a combustion product; (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction; (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂); (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂; (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂; (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂; (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂; (h) separating at least a portion of the second gas stream into an ethylene stream and a third gas stream, wherein the third gas stream comprises unconverted hydrocarbons, CO, and H₂; and (i) introducing at least a portion of the first gas stream and/or at least a portion of the third gas stream to a second reaction zone to produce methanol and a second fuel gas stream.

A second aspect, which is the method of the first aspect further comprising compressing at least a portion of the pyrolysis reaction product to a first pressure range of about 150 psig to about 300 psig prior to the step (d) of introducing the pyrolysis reaction product to a carbon dioxide removal unit.

A third aspect, which is the method of the second aspect further comprising compressing at least a portion of the second portion of the CO₂ free product stream to a second pressure range of about 200 psig to about 350 psig prior to the step (f) of contacting the acetylene solution with a second portion of the CO₂ free product stream.

A fourth aspect, which is the method of any one of the first through the third aspects, wherein at least a portion of the aprotic polar solvent stream is recycled to the acetylene absorption unit.

A fifth aspect, which is the method of any one of the first through the fourth aspects, wherein the aprotic polar solvent stream comprises N-methyl-2-pyrrolidone, dimethylformamide, acetone, or combinations thereof.

A sixth aspect, which is the method of any one of the first through the fifth aspects, wherein a portion of the first gas stream, a portion of the third gas stream, at least a portion of the second fuel gas stream, or combinations thereof is (i) recycled to the combustion zone as the first fuel gas stream; and/or (ii) used as a fuel stream other than the first fuel gas stream.

A seventh aspect, which is the method of any one of the first through the sixth aspects, wherein the first reactant mixture is characterized by a temperature of equal to or greater than about 700° C.

An eighth aspect, which is the method of any one of the first through the seventh aspects, wherein the first reaction zone is characterized by a residence time of from about 0.1 milliseconds (ms) to about 100 ms.

A ninth aspect, which is the method of any one of the first through the eighth aspects, wherein the first gas stream is characterized by a H₂/CO molar ratio of from about 1.5:1 to about 3.0:1.

A tenth aspect, which is the method of any one of the first through the ninth aspects, wherein the second gas stream is characterized by a H₂/CO molar ratio of from about 0.5:1 to about 1.5:1.

An eleventh aspect, which is the method of any one of the first through the tenth aspects further comprising (i) compressing at least a portion of the first gas stream and/or at least a portion of the third gas stream to a third pressure range of about 1,000 psig to about 1,300 psig to produce a compressed gas stream; and (ii) introducing at least a portion of the compressed gas stream to the second reaction zone.

A twelfth aspect, which is the method of the eleventh aspect, wherein the second reaction zone has an M ratio requirement of from about 2.0 to about 2.2; wherein the compressed gas stream is characterized by an M ratio of from about 2.0 to about 2.2; and wherein the M ratio is a molar ratio defined as (H₂—OO₂)/(CO+CO₂).

A thirteenth aspect, which is the method of the eleventh aspect, wherein the compressed gas stream is characterized by an M ratio other than from about 2.0 to about 2.2, wherein the M ratio is a molar ratio defined as (H₂—CO₂)/(CO+CO₂), and wherein at least a portion of the compressed gas stream is subjected to a water-gas shift reaction to produce a shifted gas stream characterized by an M ratio of from about 2.0 to about 2.2.

A fourteenth aspect, which is the method of the thirteenth aspect, wherein at least a portion of the shifted gas stream is introduced to the second reaction zone.

A fifteenth aspect, which is the method of any one of the first through the fourteenth aspects, wherein the first fuel gas stream and the hydrocarbon stream are the same or different.

A sixteenth aspect, which is the method of any one of the first through the fifteenth aspects, wherein the hydrocarbon stream comprises methane, natural gas, natural gas liquids, associated gas, well head gas, enriched gas, higher hydrocarbons, paraffins, olefins, alcohols, oxygenates, C₁ to C₆ compounds, or combinations thereof.

A seventeenth aspect, which is the method of any one of the first through the sixteenth aspects, wherein the oxidant gas comprises oxygen, purified oxygen, air, oxygen-enriched air, or combinations thereof.

An eighteenth aspect, which is the method of any one of the first through the seventeenth aspects, wherein the second reaction zone comprises a catalyst comprising Cu, Cu/ZnO, Cu/ThO₂, Cu/Zn/Al₂O₃, Cu/ZnO/Al₂O₃, Cu/Zr, or combinations thereof.

A nineteenth aspect, which is the method of any one of the first through the eighteenth aspects, wherein the first gas stream comprises H₂ in an amount of from about 40 mol % to about 60 mol %.

A twentieth aspect, which is a method for producing ethylene and hydrogen comprising (a) introducing a first fuel gas stream and an oxidant gas to a combustion zone to produce a combustion product; (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction; (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂); (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂; (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂; (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂; (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂; (h) separating at least a portion of the second gas stream into an ethylene stream and a third gas stream, wherein the third gas stream comprises unconverted hydrocarbons, CO, and H₂; and (i) introducing at least a portion of the first gas stream and/or at least a portion of the third gas stream to a pressure swing adsorption (PSA) unit to produce a hydrogen stream and a PSA fuel gas product stream.

A twenty-first aspect, which is the method of the twentieth aspect, wherein the third gas stream comprises H₂ in an amount of from about 25 mol % to about 40 mol %.

A twenty-second aspect, which is the method of any one of the twentieth and the twenty-first aspects, wherein a portion of the first gas stream, a portion of the third gas stream, at least a portion of the PSA fuel gas product stream, or combinations thereof is (i) recycled to the combustion zone as the first fuel gas stream; and/or (ii) used as a fuel stream other than the first fuel gas stream.

A twenty-third aspect, which is a method for producing ethylene, methanol and hydrogen, the method comprising (a) introducing a first fuel gas stream and an oxidant gas to a combustion zone to produce a combustion product; (b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction; (c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂); (d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂; (e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂; (f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂; (g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂; (h) separating at least a portion of the second gas stream into an ethylene stream and a third gas stream, wherein the third gas stream comprises unconverted hydrocarbons, CO, and H₂; (i) introducing a first portion of the combined first gas stream and third gas stream to a second reaction zone to produce methanol and a second fuel gas stream; and (j) introducing a second portion of the combined first gas stream and third gas stream to a pressure swing adsorption (PSA) unit to produce a hydrogen stream and a PSA fuel gas product stream.

A twenty-fourth aspect, which is the method of the twenty-third aspect further comprising recycling a portion of the first gas stream, a portion of the third gas stream, at least a portion of the second fuel gas stream, at least a portion of the PSA fuel gas product stream, or combinations thereof to the combustion zone as the first fuel gas stream.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. A method for producing ethylene and methanol comprising:
(a) introducing a first fuel gas stream and an oxidant gas to a combustion zone to produce a combustion product;
(b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction;
(c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂);
(d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂;
(e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂;
(f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂;
(g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂;
(h) separating at least a portion of the second gas stream into an ethylene stream and a third gas stream, wherein the third gas stream comprises unconverted hydrocarbons, CO, and H₂; and
(i) introducing at least a portion of the first gas stream and/or at least a portion of the third gas stream to a second reaction zone to produce methanol and a second fuel gas stream.

2. The method of claim 1 further comprising compressing at least a portion of the pyrolysis reaction product to a first pressure range of about 150 psig to about 300 psig prior to the step (d) of introducing the pyrolysis reaction product to a carbon dioxide removal unit.

3. The method of claim 2 further comprising compressing at least a portion of the second portion of the CO₂ free product stream to a second pressure range of about 200 psig to about 350 psig prior to the step (f) of contacting the acetylene solution with a second portion of the CO₂ free product stream.

4. The method of claim 1, wherein at least a portion of the aprotic polar solvent stream is recycled to the acetylene absorption unit.

5. The method of claim 1, wherein the aprotic polar solvent stream comprises N-methyl-2-pyrrolidone, dimethylformamide, acetone, or combinations thereof.

6. The method of claim 1, wherein a portion of the first gas stream, a portion of the third gas stream, at least a portion of the second fuel gas stream, or combinations thereof is (i) recycled to the combustion zone as the first fuel gas stream; and/or (ii) used as a fuel stream other than the first fuel gas stream.

7. The method of claim 1, wherein the first reactant mixture is characterized by a temperature of equal to or greater than about 700° C.

8. The method of claim 1, wherein the first reaction zone is characterized by a residence time of from about 0.1 milliseconds (ms) to about 100 ms.

9. The method of claim 1, wherein the first gas stream is characterized by a H₂/CO molar ratio of from about 1.5:1 to about 3.0:1.

10. The method of claim 1, wherein the second gas stream is characterized by a H₂/CO molar ratio of from about 0.5:1 to about 1.5:1.

11. The method of claim 1 further comprising (i) compressing at least a portion of the first gas stream and/or at least a portion of the third gas stream to a third pressure range of about 1,000 psig to about 1,300 psig to produce a compressed gas stream; and (ii) introducing at least a portion of the compressed gas stream to the second reaction zone.

12. The method of claim 11, wherein the second reaction zone has an M ratio requirement of from about 2.0 to about 2.2; wherein the compressed gas stream is characterized by an M ratio of from about 2.0 to about 2.2; and wherein the M ratio is a molar ratio defined as (H₂—CO₂)/(CO+CO₂).

13. The method of claim 11, wherein the compressed gas stream is characterized by an M ratio other than from about 2.0 to about 2.2, wherein the M ratio is a molar ratio defined as (H₂—CO₂)/(CO+CO₂), and wherein at least a portion of the compressed gas stream is subjected to a water-gas shift reaction to produce a shifted gas stream characterized by an M ratio of from about 2.0 to about 2.2.

14. The method of claim 13, wherein at least a portion of the shifted gas stream is introduced to the second reaction zone.

15. The method of claim 1, wherein the hydrocarbon stream comprises methane, natural gas, natural gas liquids, associated gas, well head gas, enriched gas, higher hydrocarbons, paraffins, olefins, alcohols, oxygenates, C₁ to C₆ compounds, or combinations thereof.

16. The method of claim 1, wherein the second reaction zone comprises a catalyst comprising Cu, Cu/ZnO, Cu/ThO₂, Cu/Zn/Al₂O₃, Cu/ZnO/Al₂O₃, Cu/Zr, or combinations thereof.

17. The method of claim 1, wherein the first gas stream comprises H₂ in an amount of from about 40 mol % to about 60 mol %.

18. A method for producing ethylene and hydrogen comprising:
(a) introducing a first fuel gas stream and an oxidant gas to a combustion zone to produce a combustion product;
(b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction;
(c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂);
(d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂;
(e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂;
(f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂;
(g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂;
(h) separating at least a portion of the second gas stream into an ethylene stream and a third gas stream, wherein the third gas stream comprises unconverted hydrocarbons, CO, and H₂; and
(i) introducing at least a portion of the first gas stream and/or at least a portion of the third gas stream to a pressure swing adsorption (PSA) unit to produce a hydrogen stream and a PSA fuel gas product stream.

19. The method of claim 18, wherein the third gas stream comprises H₂ in an amount of from about 25 mol % to about 40 mol %.

20. A method for producing ethylene, methanol and hydrogen, the method comprising:
(a) introducing a first fuel gas stream and an oxidant gas to a combustion zone to produce a combustion product;
(b) introducing a first reactant mixture to a first reaction zone, wherein the first reactant mixture comprises a hydrocarbon stream and at least a portion of the combustion product, wherein the hydrocarbon stream comprises natural gas and/or higher hydrocarbons, and wherein the combustion product heats the hydrocarbon stream to a temperature effective for a pyrolysis reaction;
(c) allowing at least a portion of the first reactant mixture to react via the pyrolysis reaction and produce a pyrolysis reaction product, wherein the pyrolysis reaction product comprises unconverted hydrocarbons, acetylene, ethylene, carbon monoxide (CO), hydrogen (H₂), water (H₂O), and carbon dioxide (CO₂);
(d) introducing at least a portion of the pyrolysis reaction product to a carbon dioxide removal unit to produce a CO₂ stream and a CO₂ free product stream, wherein the CO₂ stream comprises CO₂ and H₂O, and wherein the CO₂ free product stream comprises unconverted hydrocarbons, acetylene, ethylene, CO, and H₂;
(e) contacting a first portion of the CO₂ free product stream with an aprotic polar solvent in an acetylene absorption unit to produce an acetylene solution and a first gas stream, wherein the aprotic polar solvent absorbs at least a portion of the acetylene of the first portion of the CO₂ free product stream to produce the acetylene solution, wherein the acetylene solution comprises at least a portion of the acetylene of the first portion of the CO₂ free product stream, and wherein the first gas stream comprises unconverted hydrocarbons, ethylene, CO, and H₂;
(f) contacting at least a portion of the acetylene solution with a second portion of the CO₂ free product stream in a liquid phase hydrogenation reactor to produce a hydrogenation product, wherein the CO₂ free product stream hydrogenates at least a portion of the acetylene of the acetylene solution to produce ethylene, wherein the hydrogenation product comprises aprotic polar solvent, unconverted hydrocarbons, ethylene, CO, and H₂;
(g) separating at least a portion of the hydrogenation product into an aprotic polar solvent stream and a second gas stream, wherein the aprotic polar solvent stream comprises at least a portion of the aprotic polar solvent of the hydrogenation product, and wherein the second gas stream comprises unconverted hydrocarbons, ethylene, CO, and H₂;
(h) separating at least a portion of the second gas stream into an ethylene stream and a third gas stream, wherein the third gas stream comprises unconverted hydrocarbons, CO, and H₂;
(i) introducing a first portion of the combined first gas stream and third gas stream to a second reaction zone to produce methanol and a second fuel gas stream; and
(j) introducing a second portion of the combined first gas stream and third gas stream to a pressure swing adsorption (PSA) unit to produce a hydrogen stream and a PSA fuel gas product stream.
